(19) United States
(12) Patent Application Publication
(10) Pub. No.: US 2009/0010858 A1
(43) Pub. Date: Jan. 8, 2009

(54) Oral cavity disinfectant and oral cavity disinfecting method

(76) Inventor: Hirofumi Asano, Tokyo (JP)

Correspondence Address: MASUVALLEY & PARTNERS, 8765 AERO DRIVE, SUITE 312, SAN DIEGO, CA, 92123, US

(21) Appl. No.: 11/728,375

(22) Filed: Jul. 2, 2007

(51) Int. Cl. A61K 8/20; A61K 8/00; A61Q 11/00

(52) U.S. Cl. 424/53; 424/49

(57) ABSTRACT

Provided is an oral cavity disinfectant prepared by using a mixed aqueous solution containing a substance for dissolving proteins deposited on the tooth surface to sterilize microbes and a calcium salt-precipitating substance, respectively, at predetermined concentrations, that can be used to remove the bio film easily in a manner similar to tooth brushing. Thus, in a first embodiment of the present invention, an oral cavity disinfectant for the removal of microbes deposited on the enamel formed on the tooth surface is provided. The oral cavity disinfectant is a mixed aqueous solution containing a protein-dissolving substance dissolving proteins deposited on the enamel surface and a calcium salt-precipitating substance, respectively, at predetermined concentrations.

### BACKGROUND OF THE INVENTION

1. Field of the Invention

The present invention relates to an oral cavity disinfectant and an oral cavity disinfecting method, and in particular, an oral cavity disinfectant and an oral cavity disinfecting method for prevention of periodontal disease and caries.

2. Description of the Related Art

Recently along with the development of oral hygiene and dental chemicals, there is certainly a trend toward a decrease of diseases caused by an unsanitary condition in the oral cavity such as caries and periodontal disease. On the other hand, diversification of life style and westernized meals are forming a situation leading to wide-spread diffusion of caries or periodontal disease. For example, diversification of life style and globalization of the society lead to indistinct labor hours and an increase in the number of companies of 24-hour shifted operation, and thus, produce an environment where foods are always available (for example, in convenience stores, mid-night restaurants, etc.). Such an environment means that foods are available anytime and thus, that the oral cavity is more frequently contaminated. In westernization of meals, served are an increased number of foods softer and higher in nutrition than traditional Japanese foods, resulting in decrease in mastication frequency and saliva quantity hygienically and thus, deterioration of the sterilization capacity in the oral cavity. High food nutrition accelerates proliferation of the microbes in the oral cavity, causing a problem that the microbes are not practically removed by tooth brushing.

As a result, recently there are an increasing number of people receiving professional mechanical tooth cleaning (PMTC). In the PMTC, tartar and tooth coloring, for example, the colorant in tooth polishing and tea incrustation are removed with an ultrasonic scaler; the bio film of microbes in the gum is broken down by ultrasonication; and the tooth is polished coarsely with a quick jet, finely with a brush, and coated with fluorine in the dental office.

In this way, the microbes and the film of organic matter deposited on a tooth hard tissue in the oral cavity are removed by mechanical tooth surface cleaning using an abrasive-containing dentifrice. In some of the dentifrices, a disinfectant is added for an improvement in effectiveness.

In the concept of mechanical tooth surface cleaning, the enamel surface is considered to be a smooth and uniform crystal of an inorganic matter. In the conventional concept of mechanical tooth surface cleaning with dentifrice, the enamel surface is considered to be a smooth and uniform crystal of an inorganic matter. The concept of mechanical tooth surface cleaning is that the microbes and other microbial adhesive components on the enamel surface are removed with an abrasive by mechanical tooth surface cleaning. Care should be given in conventional methods because the hard tissue on the tooth surface is also polished with an abrasive.

A prior art, Japanese Patent Application Laid-Open (JP-A) No. 2003-200174, discloses a sterile water producing apparatus for producing sterile water by mixing an aqueous acid solution of hydrochloric acid, acetic acid, or a mixture thereof and an aqueous chlorine-based solution such as sodium hypochlorite, chlorine dioxide, or a mixture thereof with water. The sterile water producing apparatus includes a first mixer for mixing the acid into water stream, [a first mixer placed downstream of the mixer above], a second mixer for mixing the chlorine-based aqueous solution into the water stream that is placed downstream of the first mixer, and [a second mixer placed downstream of the second mixer above]. JP2003-200174A also discloses a method of producing sterile water including the steps performed by the apparatus, and a dental polishing device using the sterile water produced by the production method.

As described above, the bio film has been known to cause periodontal disease and caries, but there has been no advantageous effect observed when the tooth is treated with an antibacterial agent for prevention of bio film deposition, and thus, the bio film should be removed with an abrasive, causing a problem of greater load on the patient.

In addition, it has not been possible to remove microbial products and components and organic components such as saliva proteins that have already penetrated into the enamel structure by the mechanical tooth surface cleaning.

The conventional method disclosed in JP2003-200174A has a problem of greater cost because it demands a large facility for production of sterile water. Although a dental polishing device using sterile water is disclosed, patients should still go to a dentist for dental sterilization, causing a problem of greater patient load.

### SUMMARY OF THE INVENTION

An object of the present invention, is to provide an oral cavity disinfectant prepared by using a mixed aqueous solution containing a calcium salt-precipitating substance and a substance dissolving proteins respectively at predetermined concentrations, that can be used to remove the bio film easily in a manner similar to tooth brushing.

Another object of the present invention is to dissolve and remove not only protein components on an enamel structure but also organic components in the enamel structure, for prevention of microbial proliferation on the tooth surface.

A first aspect of the present invention, which has been made to solve the problems above, is an oral cavity disinfectant for removing bio film microbes formed on a hard tissue in an oral cavity surface and a nutritional source thereof, comprising a calcium salt-precipitating substance and a protein-dissolving substance for dissolving proteins deposited on the surface respectively at predetermined concentrations.

The main components of the present invention are a protein-dissolving substance and a calcium salt-precipitating substance. There are many proteins deposited on the surface of the hard tissue in an oral cavity, and microbes deposit on the tooth surface via the proteins and proliferate by using the proteins as a nutrient source. Accordingly, it is necessary to remove the proteins serving as the nutrient source for the microbes. The bio film is generally removed mechanically, and there is almost no report that the bio film is removed chemically and the proteins are also dissolved and removed. In the present invention, the bio film formed on the surface of the hard tissue in an oral cavity is dissolved with sodium hypochlorite and the proteins on the hard tissue surface are dissolved chemically, while the calcium salt is allowed to precipitate, by the latter method.

The calcium salt-precipitating substance is sodium hydroxide or sodium bicarbonate and the protein-dissolving substance is sodium hypochlorite.

In the present invention, the cubic structure of protein formed on the surface of the hard tissue in the oral cavity is dissolved with sodium hypochlorite; the calcium salts deposited on a solid phase in the oral cavity are allowed to precipitate with sodium hydroxide or sodium bicarbonate; and the oral cavity is disinfected with sodium hypochlorite.

The oral cavity disinfectant is a mixture of sodium hypochlorite at a concentration of 100 ppm to 200 ppm and sodium hydroxide at a concentration of 150 ppm to 300 ppm.

Safety of the oral cavity disinfectant according to the present invention is most critical because it is used in the oral cavity. Thus, the concentration of sodium hypochlorite was determined by measuring the concentration at which the bio film is decomposed most rapidly under a phase-contrast microscope, and the concentration of sodium hydroxide was so determined that the pH of the disinfectant becomes almost the same as or lower than that of the alkaline hot springs in Japan. As a result, the optimum concentration of sodium hypochlorite was found to be in the range of 100 ppm to 200 ppm. The favorable concentration of sodium hydroxide is found to be in the range of 150 ppm to 300 ppm both from the point of sense of use and safety.

The oral cavity disinfectant contains a predetermined amount of flavoring agent.

Sodium hypochlorite is generated by supplying chlorine into an aqueous sodium hydroxide. As a result, the solution stored in a sealed container generates chlorine odor when a cap of the container is removed. There are many people who dislike the chlorine odor, although the degree depends on the individual. Thus in the present invention, a predetermined amount of flavoring agent is added to the oral cavity disinfectant for reduction of the chlorine odor.

A second aspect of the present invention relates to an oral cavity disinfecting method for sterilizing microbes in an oral cavity, by placing a disinfectant in the form of a paste, gel, foam, or liquid prepared by blending the oral cavity disinfectant according to the first aspect with predetermined amounts of various additives on a toothbrush and cleaning the tooth surface and gum by abrasion.

Most of the bio film can be removed by the conventional PMTC method. According to the present invention, it is possible to remove the bio film deposited in a solid phase in the oral cavity by brushing the tooth with a toothbrush carrying the oral cavity disinfectant according to the present invention containing predetermined amounts of various additives in the form of a paste, gel, foam, or liquid in a period shorter than that by PMTC, and thus, the method demands no additional special expensive devices.

According to the first aspect of the invention, it is possible to prevent microbial proliferation after cleaning by dissolving proteins to remove a microbial nutrient source, with the oral cavity disinfectant, i.e., a mixed solution containing a protein-dissolving substance dissolving the proteins deposited on the surface of the hard tissue in the oral cavity and a calcium salt-precipitating substance respectively at predetermined concentrations.

Further, it is possible to prepare the aqueous solutions easily miscible and higher in safety because the calcium salt-precipitating substance is sodium hydroxide or sodium bicarbonate and the protein-dissolving substance is sodium hypochlorite.

Furthermore, it is possible to use the oral cavity disinfectant favorably both from the point of sense of use and safety because the oral cavity disinfectant is a mixed aqueous solution containing sodium hypochlorite at a concentration of 100 ppm to 200 ppm and sodium hydroxide at a concentration of 150 ppm to 300 ppm.

Moreover, it is possible to reduce chlorine odor and improve the sense of use because a predetermined amount of flavoring agent is added to the oral cavity disinfectant.

According to the second aspect, it is possible to remove the bio film efficiently because the bio film in the oral cavity is dissolved and the microbes therein are sterilized by cleaning the tooth surface and gum with a toothbrush carrying the oral cavity disinfectant according to the first aspect.

### BRIEF DESCRIPTION OF THE DRAWINGS

FIG. 1 is an electron micrograph showing the enamel surface;

FIGS. 2A and 2B are diagrams illustrating an electrostatic bonding pattern of a microbe to an acquired pellicle and an epithelial cell;

FIGS. 3A and 3B are diagrams showing data on results of a saliva test concerning microbes in the oral cavity of subject 1 before use;

FIGS. 4A and 4B are diagrams showing data on results of a saliva test concerning microbes in the oral cavity of subject 1 after use;

FIGS. 5A and 5B are diagrams showing data on results of a saliva test concerning microbes in the oral cavity of subject 2 before use;

FIGS. 6A and 6B are diagrams showing data on results of a saliva test concerning microbes in the oral cavity of subject 2 after use;

FIGS. 7A and 7B are diagrams showing data on results of a saliva test concerning microbes in the oral cavity of subject 3 before use;

FIGS. 8A and 8B are diagrams showing data on results of a saliva test concerning microbes in the oral cavity of subject 3 after use;

FIGS. 9A and 9B are diagrams showing data on results of a saliva test concerning microbes in the oral cavity of subject 4 before use;

FIGS. 10A and 10B are diagrams showing data on results of a saliva test concerning microbes in the oral cavity of subject 4 after use;

FIGS. 11A and 11B include a bar chart showing the change of total microbial count in the oral cavity over time and a table showing the decrease in the absolute values of the total microbial counts in the oral cavity of each subject before and after use;

FIG. 12 is a chart showing the change in the average of the total microbial counts in the oral cavity of all subjects before and after use; and

FIGS. 13A and 13B are a photograph and a diagram showing results of a mouse acute toxicity test when an oral cavity disinfectant according to the present invention is used by a mouse.

### DESCRIPTION OF THE PREFERRED EMBODIMENTS

Hereinafter, the present invention will be described in detail with reference to embodiments shown in the drawings. However, the components, kinds, combination, shape, and relative location described in the embodiment are not aimed at restricting the scope of the present invention, and are examples in description, unless specified otherwise.

The enamel structure and deposition of microbes on the enamel structure will be described before description of the embodiments of the present invention.

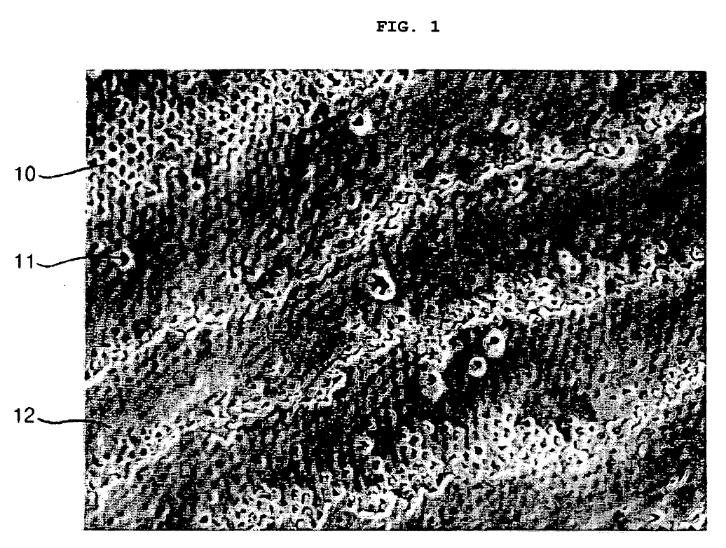

FIG. 1 is an electron micrograph showing the surface of the enamel structure. The enamel surface may be flat by observation with naked eyes, but, as shown in FIG. 1, it has pillars 10 called prismata adamantina on the surface and additionally large surface irregularity 12 and pores 11 on the surface. Thus, the enamel surface is a place, like a nest, suitable for microbes to proliferate. More specifically, it is reasonable to consider on the basis of the basic odontology that the enamel structure is a layered structure having microscopic surface irregularities and the pores are formed by a decalcification action of an organic acid generated by microbial metabolism that contain various organic matters present in the oral cavity that have penetrated there.

An energy source for microbes growing on the tooth surface is currently considered to be saliva. It is postulated in the present invention that the microbes proliferate not only on saliva but also on the organic matter deposited on the tooth surface. Observation, for example, under an electron microscope shows that the surface layer of the enamel structure does not practically have a simple structure, but has a characteristic complicated fine structure histologically different in calcification degree. The fine structure is further fissured by decalcification by the organic acid generated by microbial metabolism in the oral cavity. Thus, the microbes may be considered to proliferate by consuming the organic matter remaining in the enamel fine structure as part to the metabolites needed for proliferation on the tooth surface.

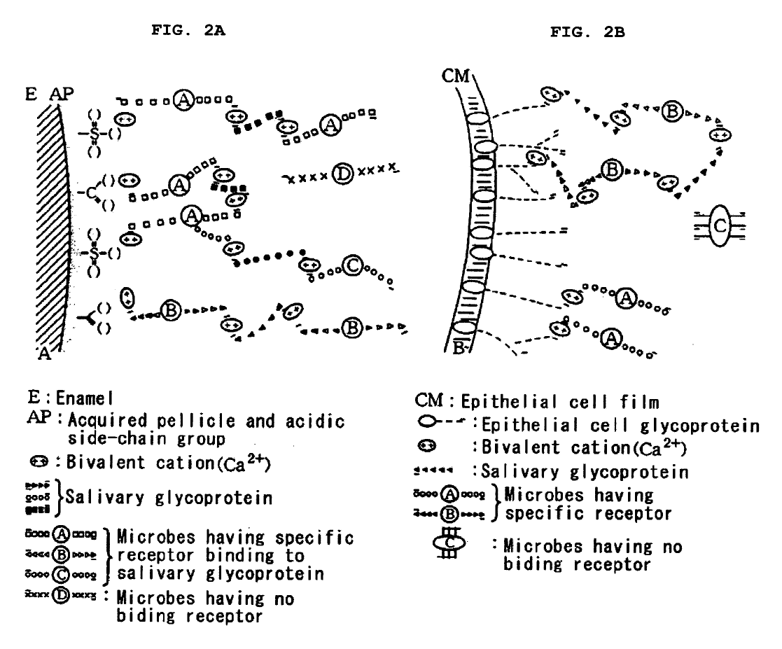

FIGS. 2A and 2B are a diagram illustrating an electrostatic bonding pattern of a microbe to an acquired pellicle and an epithelial cell. FIG. 2A is a chart illustrating the electrostatic bonding pattern of the microbe to the acquired, pellicle; and FIG. 2B is a chart illustrating the electrostatic bonding pattern of the microbe to the epithelial cell (“Dental Microbiology”, 4th Ed., Ishiyaku Publishers, Inc.).

As shown in FIG. 2A, a specific substance in salivary glycoproteins deposits on the tooth surface, i.e., a hydroxyapatite (HA) layer, thereby forming a pellicle (acquired thin film) in advance to microbes. The acidic side-chain group on the pellicle surface is negatively charged. Microbes are seemingly adsorbed on the tooth surface by a crosslinking of the negatively charged surfaces particularly with Ca²⁺ in saliva.

As shown in FIG. 2B, the microbes seem to be adsorbed on the tooth surface with insufficient pellicle formation directly via the Ca²⁺ of HA molecules. When in the state of polysaccharides such as glucan binding to or coating the microbe surface layer, these polysaccharide molecules and pellicles may be adsorbed on the tooth surface via hydrogen bonds. Adsorption thereof on the mucous membranes also seems to occur by an electrical interaction similar to that of the pellicle because the glycoproteins in the epithelial cell are charged electrostatically negatively.

### Embodiment of the Invention

As described above in FIG. 1, the enamel surface is not flat at all, and thus, microbes can bind thereto in the electrostatic bonding pattern described in FIG. 2. Thus in a first embodiment of the present invention, an oral cavity disinfectant for removal of a bio film formed on the tooth surface is provided. The oral cavity disinfectant is an aqueous solution containing a protein-dissolving substance dissolving the bio film deposited on the solid phase surface in an oral cavity and a pellicle protein serving as an adhesive between the solid phase and microbe and a calcium-salt-precipitating substance respectively at predetermined concentrations.

Saliva proteins are always in contact with the tooth surface. Saliva contains many proteins, and accordingly, a protein called acquired pellicle deposits on the tooth enamel structure. Microbes adhere to the surface of a hard tissue via the protein. Because the microbes proliferate on the amino acids derived from saliva proteins present on the surface of the tooth as a nutrient source, it is necessary to eliminate the proteins, the nutrient source for the microbes, from the surface of the hard tissue in the oral cavity. A method of removing the bio film mechanically (PMTC) is commonly used currently for removal of the microbes on the tooth surface, and there has been almost no report on a method of dissolving the bio film chemically, and dissolving and eliminating the proteins on the hard tissue surface additionally. In the present invention, a substance dissolving the bio film and the proteins chemically by the latter method is used as the oral cavity disinfectant. Thus by dissolving proteins and removing an adhesion factor of the microbes and a nutrient source, it is possible to prevent proliferation of microbes in the oral cavity.

In a second embodiment of the present invention, sodium hydroxide or sodium bicarbonate is used as the calcium salt-precipitating substance, and sodium hypochlorite is used as the substance dissolving proteins and sterilizing the oral cavity. It is possible to prepare aqueous solutions readily miscible with each other and superior in stability.

Both of the pharmaceutical components, sodium hypochlorite and sodium hydroxide, have an organic matter-dissolving action. Sodium hydroxide plays a role to stabilize sodium hypochlorite and also to precipitate the protein-crosslinking factor Ca²⁺ with a strong alkali such as Ca(OH)₂ or Ca (HCO₃)₂. Other bivalent cations needed for metabolism of microbes seem to be eliminated at the same time. OH⁻ in the disinfectant may be effective in artificial remineralization of the tooth surface and reducing organic components.

Ca²⁺ in saliva is active in both microbe- and protein-crosslinking factors and plays an important role in fixing proteins on the tooth surface. Removal of Ca²⁺ with a strong alkali results in a decrease in the amount of proteins and microbes deposited on the tooth surface.

Ca²⁺ is present as a protein-crosslinking factor in the oral cavity. It binds to F⁻ during treatment for prevention of dental caries by tooth-surface application of fluorine, thereby inhibiting formation of fluoroapatite on the tooth surface.

In a third embodiment of the present invention, the oral cavity disinfectant according to the present invention is a mixed aqueous solution containing sodium hypochlorite at a concentration of 100 ppm to 200 ppm and sodium hydroxide at a concentration of 150 ppm to 300 ppm. The concentration of sodium hypochlorite was determined by observing the concentration at which the bio film is decomposed most easily under a phase-contrast microscope, and the concentration of sodium hydroxide was set to a pH equal to or less than that of an alkaline hot spring in Japan. As a result, the most favorable concentration of sodium hypochlorite was found to be in the range of 100 ppm to 200 ppm. The favorable concentration of sodium hydroxide is found to be in the range of 150 ppm to 300 ppm both from the point of sense of use and safety.

A fourth embodiment of the present invention provides a method of using the oral cavity disinfectant according to the present invention, characterized by sterilizing microbes in an oral cavity by rubbing the tooth surface and gum with a toothbrush carrying the disinfectant. Although it is possible to remove most bio film by the conventional PMTC method, it is possible, in the present invention, to dissolve the surface bio film and also to remove the protein film, both a microbe adhesion factor and a nutrient source, by brushing teeth with a toothbrush carrying the oral cavity disinfectant according to the present invention. In this way, it is possible to remove the bio film efficiently and prevent regeneration of the bio film.

In a fifth embodiment of the present invention, the oral cavity disinfectant contains a predetermined amount of flavoring agent. Sodium hypochlorite is generated when chlorine is supplied into an aqueous sodium hydroxide solution. Accordingly, when a cap is removed from a tightly sealed container, chlorine odor is noticed. The chlorine odor is annoying although the degree depends on the individual. Thus in the present invention, a predetermined amount of flavoring agent is added to the oral cavity disinfectant for reduction of the chlorine odor. It is thus possible to reduce chlorine odor and improve the sense of use. Although oral cavity disinfectant has been described in the embodiments above, the oral cavity disinfectant may be used in the form of paste, gel, foam, or liquid, as it is blended with predetermined amounts of various additives.

### EXAMPLES

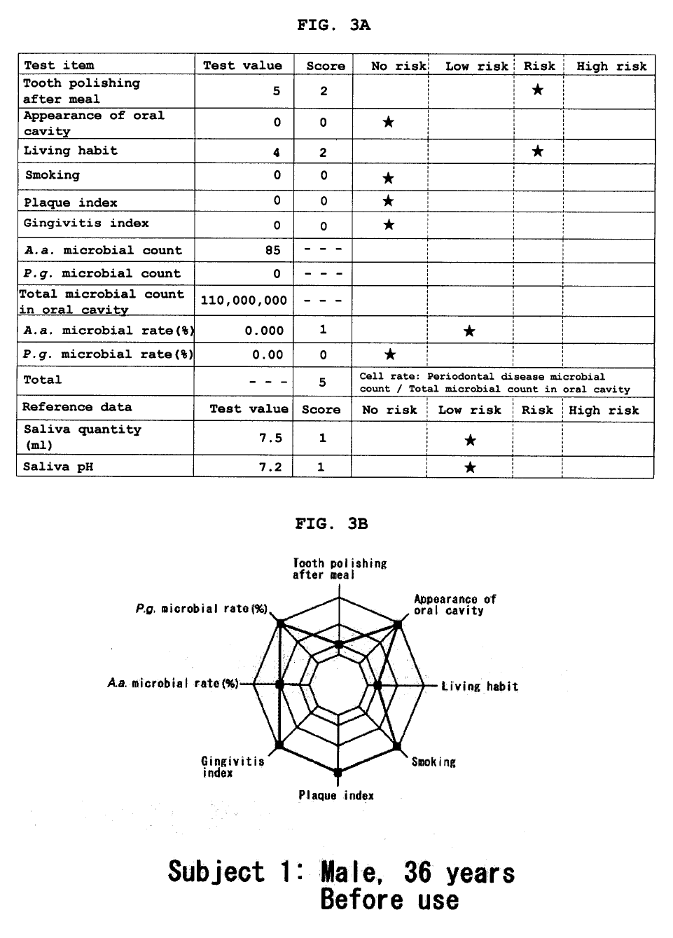

FIGS. 3 to 10 are diagrams showing data on test results of salivary microbes in an oral cavity of each subject before and after use. Table A in each figure shows the results in respective test items, and chart B shows the results in a diagram format. The test was performed by using a commercially available kit manufactured by BML Co., Ltd. Among the subjects in the Example, subject 1 is a male of 36 years; subject 2 is a female of 35 years; subject 3 is a male of 32 years; and subject 4 is a female of 31 years of age. The test items include degree of tooth polishing after meal (by organoleptic test), appearance of an oral cavity (by visual observation), living habits (meal period, foods served, etc.), smoking (smoking or non-smoking, number of cigarettes), plaque index (indicator of the amount of plaques), gingivitis index (indicator of the degree of gingivitis), A. a. microbial count, P. g. microbial count, total microbial count in the oral cavity (count of all microbes, independent of the kind), A. a. microbial rate (number of A. a. microbes in the oral cavity), P. g. microbial rate (number of P. g. microbes in the oral cavity), and saliva quantity and saliva pH as reference data. The results of these test items are digitalized and expressed as scores. The degrees of risk were divided into four grades: no risk (score: 0), low risk (score 1), normal risk (score: 2), and high risk (score: 3).

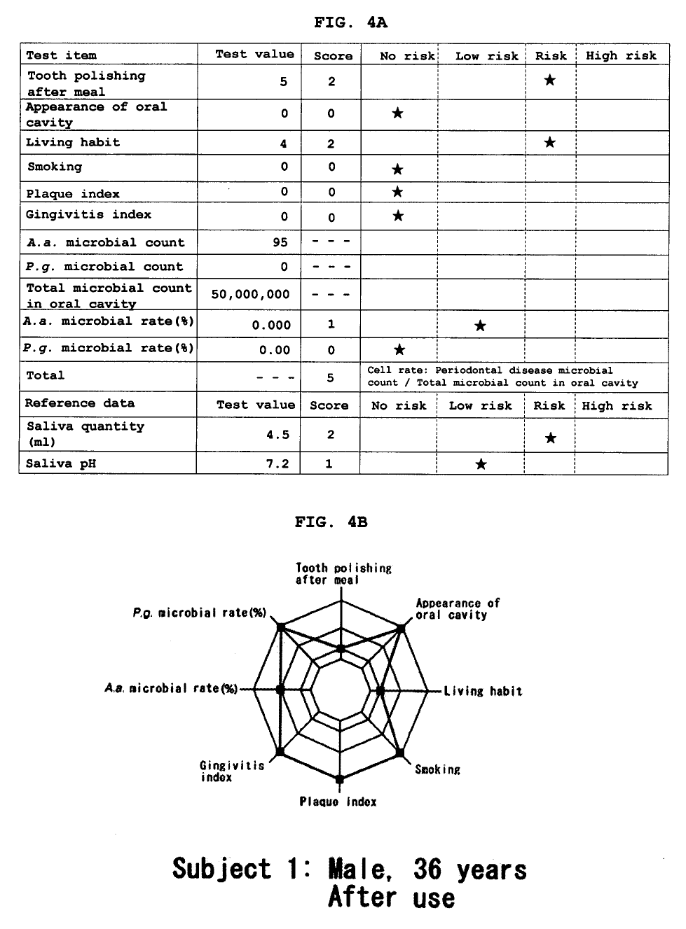

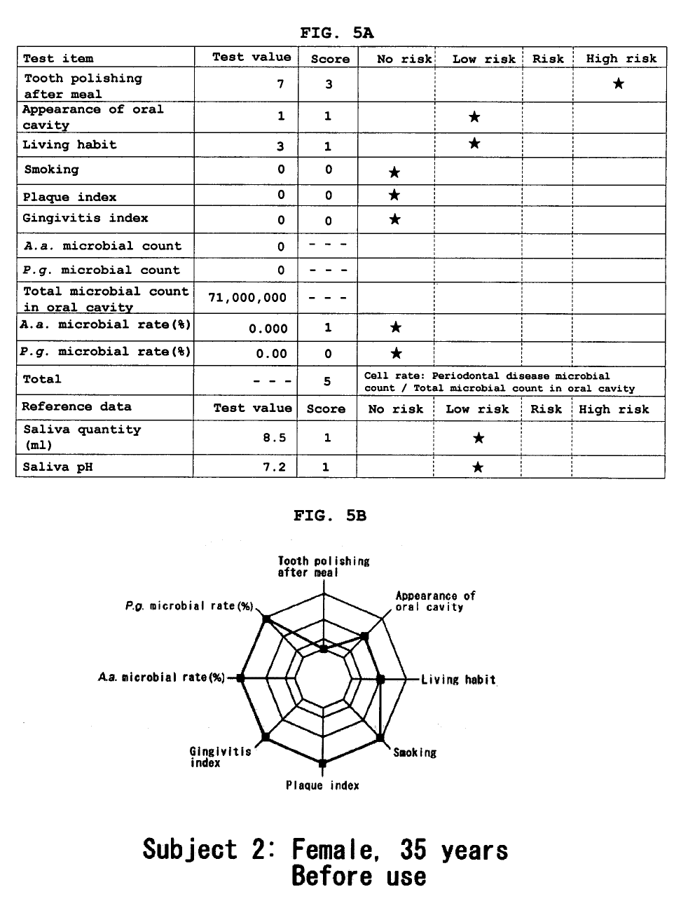

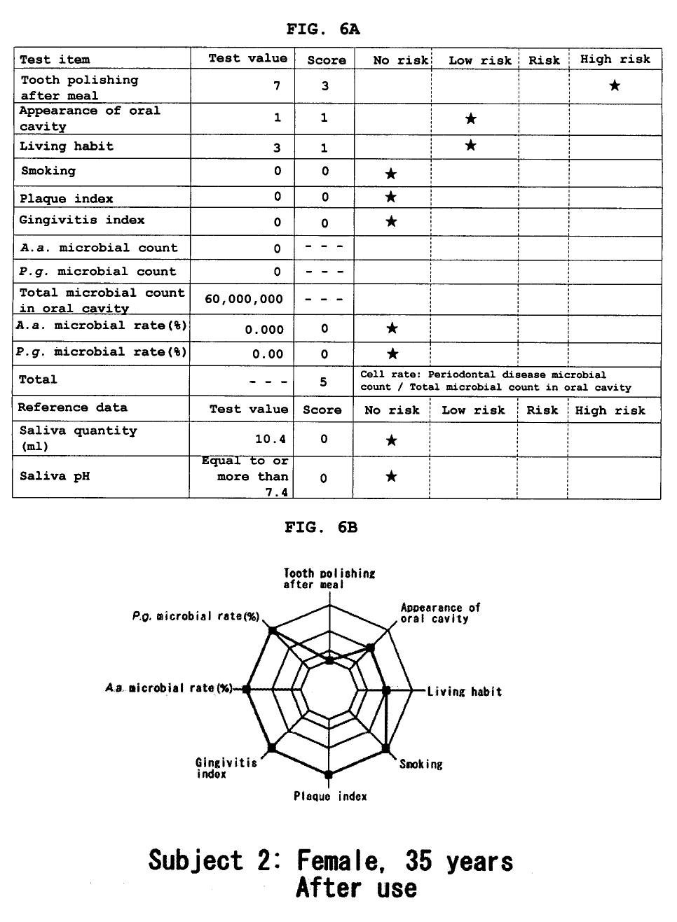

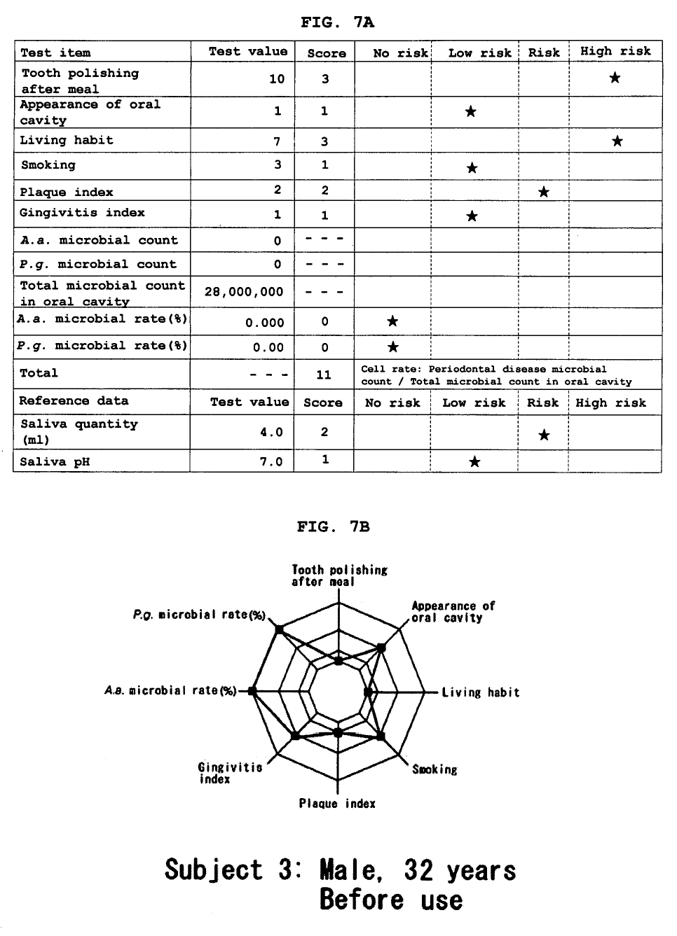

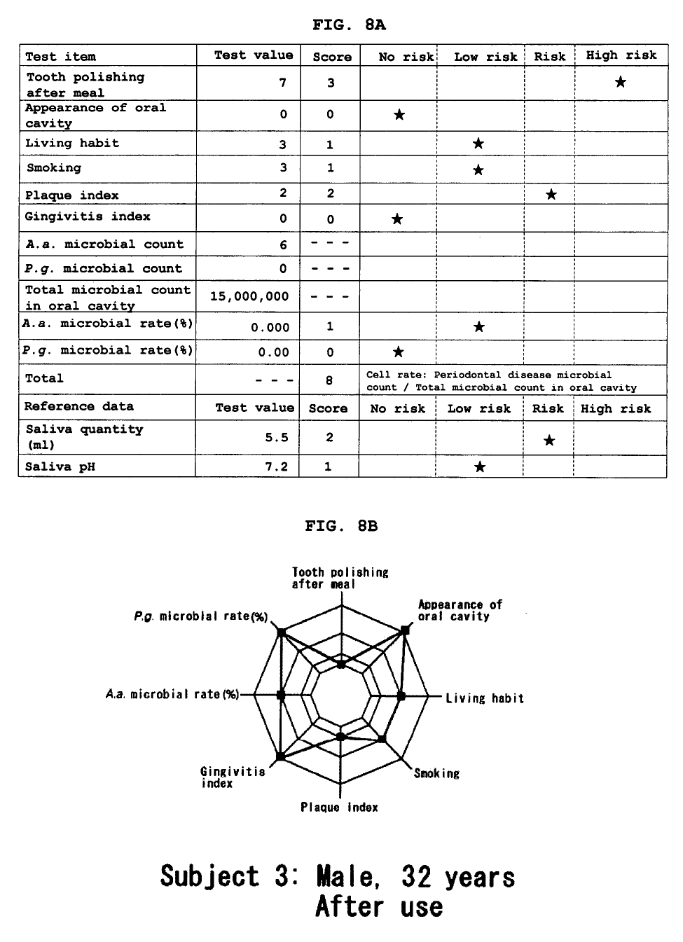

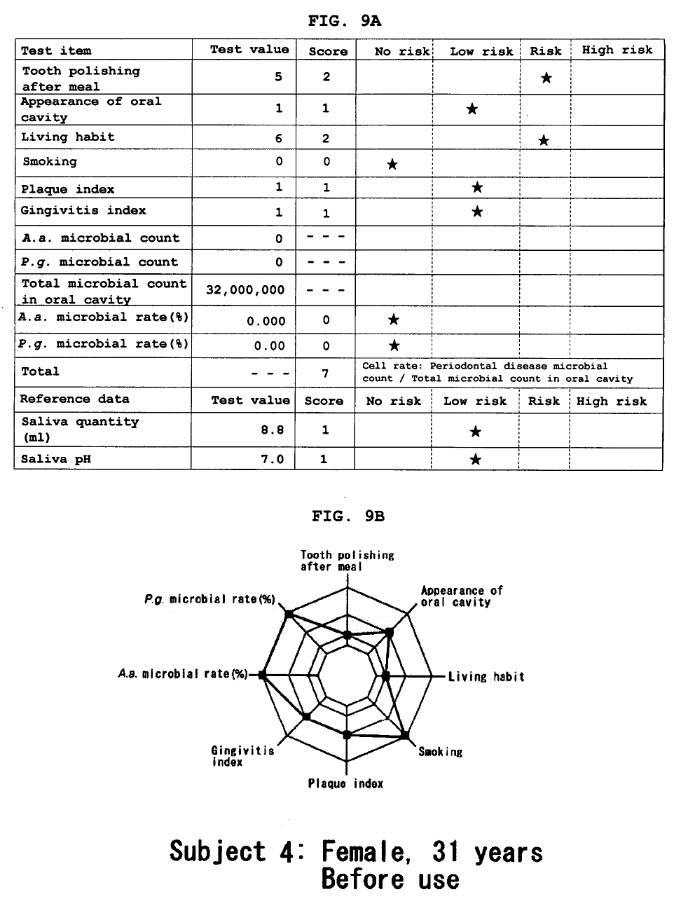

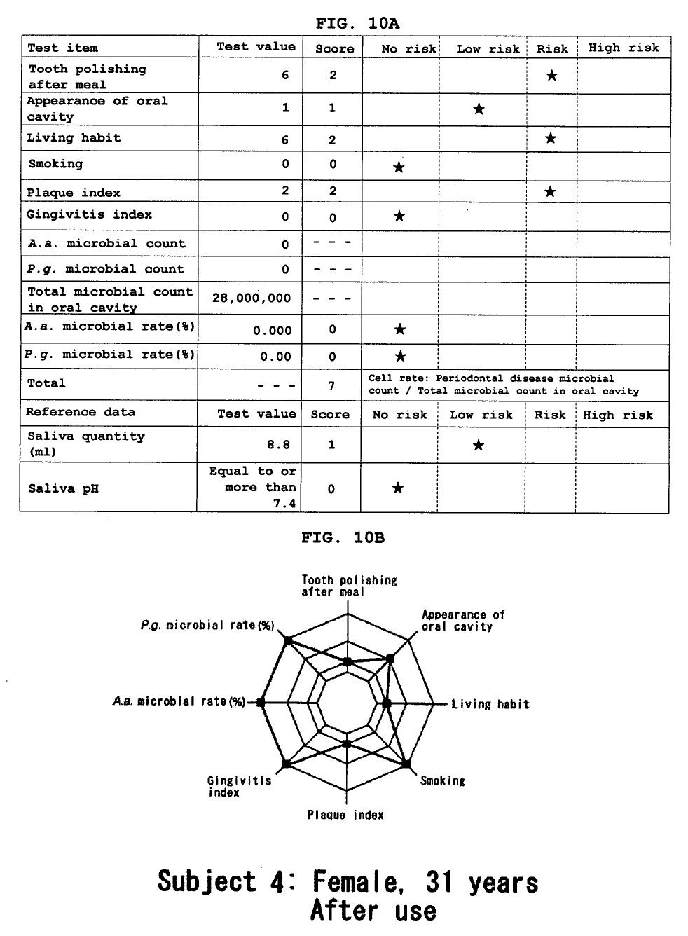

FIGS. 3 and 4 show the data of subject 1 before use (of the oral cavity disinfectant according to the present invention) and after use (of the oral cavity disinfectant according to the invention for one week). FIGS. 5 and 6 show the data of subject 2 before use (of the oral cavity disinfectant according to the invention) and after use (of the oral cavity disinfectant according to the invention for one week). FIGS. 7 and 8 show the data of subject 3 before use (of the oral cavity disinfectant according to the invention) and after use (of the oral cavity disinfectant according to the invention for one week). FIGS. 9 and 10 show the data of subject 4 before use (of the oral cavity disinfectant according to the invention) and after use (of the oral cavity disinfectant according to the invention for one week).

For example, the data of subject 1 before use will be considered below with reference to FIG. 3. The degree of tooth polishing after meals is not favorable with the subject 1, and the test value is “5”; the score is “2”; and the degree of the risk is medium. As for the appearance of the oral cavity, the test value is “0”; the score is “0”; and the risk is no risk. As for the living habit, the test value is “4”; the score is “2”; and the degree of the risk is medium. As for the smoking, the test value is “0”, the score is “0”; and the risk is no risk. As for plaque index, the value is “0”; the score is “0”; and the risk is no risk. As for the gingivitis index, the test value is “0”; the score is “0”; and the risk is no risk. As for the A. a. microbial count, the test value is “85”. As for the P. g. microbial count, the test value is “0”. As for the total microbial count in the oral cavity, the test value is “110,000,000”. The A. a. microbial rate is negligible compared to the total microbial count in the oral cavity, and thus, the test value is “0”; the score is “1”; and the risk is low risk. The P. g. microbial rate is “0”, and thus, the test value is “0”; the score is “0”; and the risk is no risk. The total risk of the subject is “5”. The amount of saliva as reference data is “7.5 ml”; the score is “1”; and the risk is low risk. The saliva pH is “7.2”; the score is “1”; and the risk is low risk.

Then, the data of subject 1 after use will be described below with reference to FIG. 4. The degree of tooth polishing after meal is not favorable, and thus, the test value is “5”; the score is “2”; and the degree of the risk is medium. As for the appearance of the oral cavity, the test value is “0”; the score is “0”; and the risk is no risk. As for the living habit, the test value is “4”; the score is “2”; and the degree of the risk is medium. As for the smoking, the test value is “0”; the score is “0”; and the risk is no risk. As for the plaque index, the test value is “0”; the score is “0”; and the risk is no risk. As for the gingivitis index, the test value is “0”; the score is “0”; and the risk is no risk. As for the A. a. microbial count, the test value is “95”. The test value of the P. g. microbial count is “0”. As for the total microbial count in the oral cavity, the test value is “50,000,000”, half of that before use. The A. a. microbial rate is negligible compared to the total microbial count in the oral cavity, and thus, the test value is “0”; but the score is “1” and the risk is low risk. The P. g. microbial rate is “0”, and thus, the test value is “0”; the score is “0”; and the risk is no risk. The total risk of the subject is “5”. The amount of saliva as reference data is “4.5 ml”; the score is “2”; and the degree of the risk is medium. The saliva pH is “7.2”, the score is “1”; and the risk is low risk.

A. a. stands for Actinobacillus actinomycetemcomitans, while P. g., for Porphyromonas gingivalis.

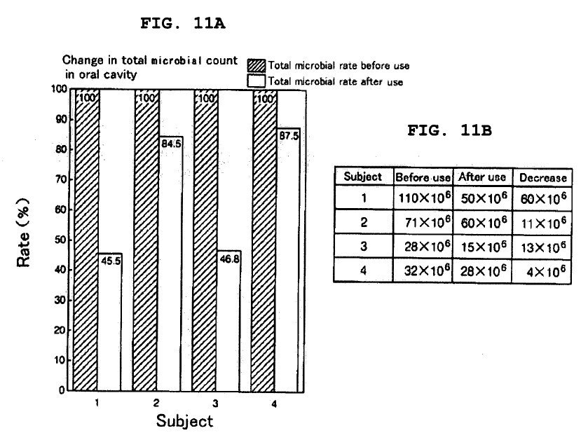

FIG. 11A is a bar chart showing the results of subjects 1 to 4 concerning the total microbial count in the oral cavity, wherein the data are normalized to 100 of the total microbial count before use. As apparent from FIG. 11A, subject 1 had the most reduced total microbial count of 45.5% after use. Subject 3 had the next most reduced microbial count of 46.8%; subject 2 had a microbial count of 84.5%; and subject 4 had a microbial count of 87.5%. There was the least effect after use with regard to subject 4.

FIG. 11B is a table showing the absolute total microbial counts in the oral cavity of each subject before and after use. The effect of the disinfectant will be considered below with reference to FIG. 11B, from the other viewpoint with these numerical values. Most important is to reduce the absolute value of the total microbial count in the oral cavity. From the viewpoint above, the total microbial count in the oral cavity of subject 3 after use is smallest at 15×10⁶, and subject 4, subject 1, and subject 2 follow in that order. Subject 3 has a smallest total microbial count in the oral cavity before use at 28×10⁶, and subjects 4, 2 and 1 follow in that order. The results indicate that it is preferable to reduce the total microbial count in the oral cavity before use, in order to reduce the absolute total microbial count in the oral cavity. In other words, it is important to keep the oral cavity clean in a daily living habit.

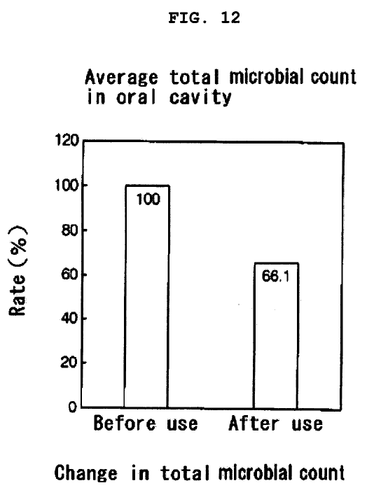

FIG. 12 is a chart showing the change in the average of the total microbial counts in the oral cavity of all subjects in FIG. 11 before and after use. The values are normalized, with respect to 100% of the average before use. As shown in the FIG. 12, the average after use is 66.1%, showing a drastic decrease in the total microbial count in the oral cavity even one week after use of the oral cavity disinfectant according to the invention. The result indicates that the advantageous effect of the oral cavity disinfectant according to the invention remains for one week or more. The oral cavity disinfectant according to the invention has been used for control of the bio film in the oral cavity, and is very superior in safety to chlorhexidine gluconate solutions which are reported to cause anaphylactic shock in several cases.

The results of the saliva test showed a decrease in the total microbial count in the oral cavity even after one week. Use of the oral cavity disinfectant seems to be more effective than conventional tooth surface cleaning methods, because it results in removal of the organic components and crosslinking factor Ca²⁺ crosslinked on the tooth surface and also of the organic matter penetrated from the tooth surface. The result is a new scientific finding.

Also newly found is that it is possible to reduce continuously the number of microbes deposited on the tooth surface by combined use of multiple food additives respectively at safe concentrations.

The disinfectant solution is neutralized rapidly by the buffering action of proteins and saliva in the oral cavity, even when it is strongly alkaline (data undisclosed).

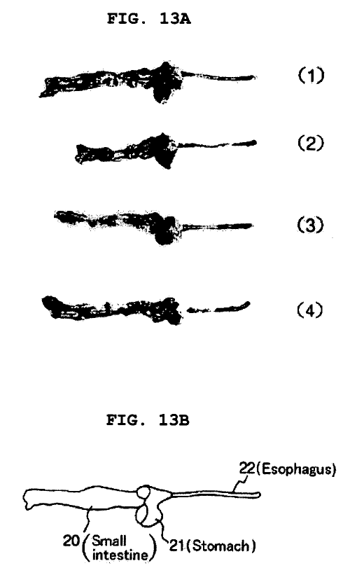

FIG. 13 is a diagram showing results of an acute toxicity test by administering the oral cavity disinfectant according to the present invention into a mouse. FIG. 13A is a diagram showing the pathologic change of mouse digestive organs when the pH of the disinfectant is changed, while FIG. 13B is a diagram showing the name of each unit.

In the test, oral cavity disinfectants different in pH were administered into a mouse, and the resulting pathologic change of the digestive organ (stomach) was observed after 1 hour. The concentration of the oral cavity disinfectant sodium hypochlorite was 100 ppm; and the pH thereof was adjusted to (1) pH 7 (physiological saline), (2) pH 9, (3) pH 11, or (4) pH 13. As a result, no pathological findings such as sore and tumor on mucous membrane of the mouse's stomach was found as shown in (1) to (4) of FIG. 13.

As described above, the oral cavity disinfectant according to the present invention, an aqueous solution containing a protein-dissolving substance dissolving the proteins deposited on the surface of the hard tissue in the oral cavity and a calcium salt-precipitating substance respectively at predetermined concentrations, removes the nutrient source for microbes by dissolving proteins, and thus, inhibits proliferation of microbes after cleaning.

The calcium salt-precipitating substance is sodium hydroxide or sodium bicarbonate and the protein-dissolving substance is sodium hypochlorite, and thus, it is possible to prepare a highly stable aqueous solution easily by mixing them together.

The oral cavity disinfectant is a mixed aqueous solution containing sodium hypochlorite at a concentration of 100 ppm to 200 ppm and sodium hydroxide at a concentration of 150 ppm to 300 ppm, and thus, can be used favorably without problems, both from the point of sense of use and stability.

The oral cavity disinfectant causes less chlorine odor and is improved in sense of use, because it contains a predetermined amount of flavoring agent.

It is also possible to dissolve the bio film and sterilize microbes in the oral cavity and thus to remove the bio film efficiently, by blending the oral cavity disinfectant according to the present invention with predetermined amounts of various additives and brushing and cleaning the tooth surface and gum with a toothbrush carrying the disinfectant in the form of a paste, gel, foam, or liquid.

Considering the anatomic fine structure of the enamel structure, the present invention is aimed at chemically dissolving and removing not only surface-bonding microbes but also the organic matter as an adhesive growth factor and the protein-crosslinking factor Ca²⁺ held deep in the fine structure on the tooth surface. Thus, the disinfectant according to the present invention enables chemical tooth-surface cleaning.

It also contains food additive-grade sodium hypochlorite and sodium hydroxide as pharmaceutical components respectively at low concentrations. There is no pharmaceutical component that causes adverse reaction and thus, the disinfectant is safe to the body.

It is also used in the form of a paste, gel, foam, or liquid as it is blended with various additives and applied on the tooth surface in the oral cavity with a toothbrush.

It is possible to remove microbes adhering in the tooth hard tissue, adhesion factors (organic matter) of microbes proliferating in the fine anatomical structure of the enamel structure, and also microbial metabolites, and to prevent reproliferation of the microbes on the tooth surface, by combined use of the oral cavity disinfectant according to the invention and a toothbrush. In addition, the disinfectant does not damage the tooth surface like mechanical tooth surface cleaning, because it contains no abrasive.

It would be possible to remove Ca²⁺ by pretreatment with the composition according to the present invention, which in turn leads to an increase in the amount of fluorine adhered to the tooth surface and prevention of dental caries.

What is claimed is:

1. An oral cavity disinfectant for removing a bio film formed on a hard tissue in an oral cavity surface and a nutrient source thereof, comprising a calcium salt-precipitating substance and a protein-dissolving substance for dissolving proteins deposited on the surface, respectively, at a group of predetermined concentrations.

2. The oral cavity disinfectant according to claim 1, wherein the calcium salt-precipitating substance is a sodium hydroxide or a sodium bicarbonate and the protein-dissolving substance is a sodium hypochlorite.

3. The oral cavity disinfectant according to claim 1 or 2, wherein the oral cavity disinfectant is a mixture of a sodium hypochlorite at a concentration of 100 ppm to 200 ppm and a sodium hydroxide at a concentration of 150 ppm to 300 ppm.

4. The oral cavity disinfectant according to any one of claims 1 to 3, wherein the oral cavity disinfectant contains a predetermined amount of flavoring agent.

5. An oral cavity disinfecting method of sterilizing a multiplicity of microbes in an oral cavity, comprising the steps of:
producing an oral cavity disinfectant comprising a calcium salt-precipitating substance comprised of a sodium hydroxide or a sodium bicarbonate and a protein-dissolving substance comprised of a sodium hypochlorite; and
placing the oral cavity disinfectant in the form of a paste, a gel, a foam, or a liquid on a toothbrush and cleaning a tooth or a gum surface.

6. The method of claim 6, further comprising the step of:
adding a predetermined amount of flavoring agent to the oral cavity disinfectant.

7. The method of claim 6, where the step of producing the oral cavity disinfectant comprising the calcium salt-precipitating substance comprised of the sodium hydroxide or the sodium bicarbonate and the protein-dissolving substance comprised of the sodium hypochlorite is performed by the step of:
mixing a sodium hypochlorite at a concentration of 100 ppm to 200 ppm and a sodium hydroxide at a concentration of 150 ppm to 300 ppm.
